(19) United States
(12) Patent Application Publication
(10) Pub. No.: US 2011/0257510 A1
(43) Pub. Date: Oct. 20, 2011

(54) SYSTEM, METHOD AND APPARATUS FOR CARDIAC INTERVENTION WITH MR STROKE DETECTION AND TREATMENT

(75) Inventor: Steffen Weiss, Hamburg (DE)

(73) Assignee: KONINKLIJKE PHILIPS ELECTRONICS N.V., EINDHOVEN (NL)

(21) Appl. No.: 13/141,091

(22) PCT Filed: Dec. 11, 2009
(86) PCT No.: PCT/IB2009/055693

§ 371 Date: Jun. 21, 2011

(30) Foreign Application Priority Data
Dec. 24, 2008 (EP) 08172889.1

(51) Int. Cl. A61B 5/055

(52) U.S. Cl. 600/411

(57) ABSTRACT

A system for the early detection and treatment of catheter-induced ischemic strokes includes a magnetic resonance system (20) and a processor (36). The magnetic resonance system includes a sequence controller for performing each of a plurality of imaging sequences and a sequence memory (32) which stores at least a magnetic resonance angiography (MRA) sequence, a diffusion-weighted imaging (DWI) sequence, and a perfusion-weighted imaging (PWI) sequence. The processor is programmed to control the magnetic resonance system to perform the steps of: performing the MRA sequence to generate a baseline MRA image; performing a catheter-tracking procedure to track a catheter; performing the DWI sequence after the catheter procedure to generate a diffusion-weighted image; performing the PWI sequence to generate a perfusion-weighted image; and combining the diffusion-weighted image and the perfusion-weighted image to generate a combined image for evaluating the ischemic stroke.

### FIELD OF THE INVENTION

The following relates to the medical arts, imaging arts, magnetic resonance arts and related arts. It finds particular application in detection of stroke and is described with particular reference thereto.

### BACKGROUND OF THE INVENTION

Strokes often represent dramatic changes in the quality of life of the patients and are associated with huge costs for rehabilitation and permanent disability. Strokes can include vessel occlusion or blockage, and also vessel rupture. Treatment of cardiovascular pathologies by transvascular catheter interventions is associated with increased stroke risk. When such interventions are performed in the left heart, the aortic arch, the carotid arteries or cerebral vasculature are exposed to intra-procedural acute ischemic stroke risk. The risk increases with procedure time and catheter size. Prevalent examples of such risks include coronary angiography, coronary balloon angioplasty, coronary stenting, carotid atherectomy, and atrial fibrillation (AF) ablation.

Because areas of the brain are damaged when deprived of blood by a stroke, speed of detection and response to such a catheter-induced stroke is the key to preventing physical harm to the patient, such as brain damage. One problem with the current stroke detection methods is that at present, there are few means for detecting these strokes. Another problem is that the risk for intra-procedural acute ischemic stroke due to AF ablation including anticoagulant treatment has been shown to be in the order of 1%. A further problem is that due to this ablation-induced stroke risk it could not yet be shown that the overall stroke risk for the patient is decreased by ablation treatment.

The present application provides an improved system, method, and apparatus, which overcomes the above-referenced problems and others.

### SUMMARY OF THE INVENTION

In accordance with one aspect, a method of early detection and treatment of catheter-induced ischemic strokes is provided. A catheter procedure is performed. A diffusion-weighted imaging (DWI) scan is performed to generate a stroke detection diffusion-weighted image. Optionally, a perfusion-weighted imaging (PWI) scan is performed to generate a stroke detection perfusion-weighted image. The stroke detection diffusion-weighted image and the stroke detection perfusion-weighted image are evaluated for a catheter-induced ischemic stroke.

In accordance with another aspect, a system for the early detection and treatment of catheter-induced ischemic strokes is provided. A magnetic resonance system includes a sequence controller for performing a plurality of imaging sequences and a sequence memory which stores at least a magnetic resonance angiography (MRA) sequence and a DWI sequence, and a PWI sequence. A processor is programmed to control the magnetic resonance system to perform the steps of: performing the MRA sequence on the head and neck to generate a baseline MRA image; performing a catheter tracking procedure to track a catheter during a catheter procedure; performing the DWI sequence to generate a stroke detection diffusion-weighted image; performing the PWI sequence to generate a stroke detection perfusion-weighted image; and combining the stroke detection diffusion-weighted image and the stroke detection perfusion-weighted image to generate a combined image for use in detecting an ischemic stroke.

One advantage of the presymptomatic detection of strokes offers the possibility to treat the stroke earlier, which is expected to preserve more brain tissue than a treatment performed after symptomatic onset.

Another advantage is that detection of such strokes provides the possibility to treat them by the administration of thrombolytics, particularly local thrombolytics.

Still further advantages and benefits will become apparent to those of ordinary skill in the art upon reading and understanding the following detailed description.

### BRIEF DESCRIPTION OF THE DRAWINGS

The invention may take form in various components and arrangements of components, and in various steps and arrangements of steps. The drawings are only for purposes of illustrating the preferred embodiments and are not to be construed as limiting the invention.

FIG. 1 illustrates a patient undergoing a catheter procedure;

FIG. 2 illustrates the method of the present application in a flow chart format.

### DETAILED DESCRIPTION OF EMBODIMENTS

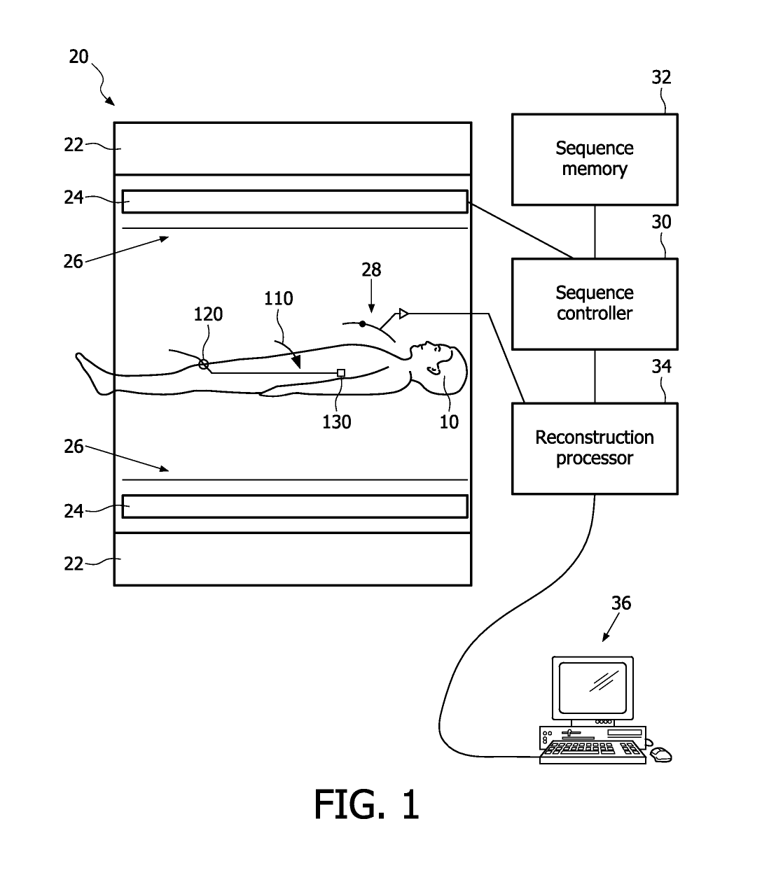

With reference to FIG. 1, a patient 10 being treated for a cardiovascular pathology is presented. The patient 10 is disposed in an imaging region of a magnetic resonance scanner 20. The scanner includes a main field magnet 22 for generating a B₀ field in the imaging region. Gradient coils 24 induce a gradient magnetic field across the B₀ field, typically along three orthogonal axes. A whole body RF coil 26 transmits RF pulses to excite and manipulate resonance in the patient in the imaging region. The whole body RF coil optionally also receives RF resonance signals from the examination region. A surface or other local RF coil 28, if present, receives the resonance signals. Optionally, the local coil can also induce and manipulate resonance. A sequence controller 30 controls the RF and gradient coils to implement a selected magnetic resonance imaging sequence. A sequence memory 32 stores DWI, PWI, MRA, and other imaging sequences. A reconstruction processor 34 reconstructs the received resonance signals into an image representation. A user interface 36 enables an operator to select imaging sequences to be performed, reconstruction operations, image formats and the like and displays reconstructed images.

The patient 10 is placed inside the MRI machine 20. A catheter 110 is inserted through a surgical sheath 120. The catheter can contain a μ-receive (micro-receive) coil 130 for purpose of catheter tracking. Likewise, during thrombolytic catheter procedure to resolve stroke. Using MR images on the monitor, the catheter is controlled to navigate the circulatory system to a location at which an intervention is to be performed. When such interventions are performed in the left heart, the aortic arch, the carotid arteries, or in cerebral vasculature, the patient is exposed to intra-procedural acute ischemic stroke risk as well as an increased risk of thrombogenicity in long (i.e., taking an extended period of time) catheter interventions. Two recognized procedural risk factors are longer procedure time and the use of larger-caliber catheters. The potential mechanisms of thrombogenesis during ablation procedures are multiple and include endothelial disruption, coagulation necrosis, electroporation injury, mechanical damage in the vessel wall, and heating of circulating blood elements by radiofrequency energy. The above mechanisms can cause activation of the cascade of events that ultimately results in thrombin generation and platelet activation. As such, the present application produces the useful, concrete and tangible result of preventing strokes.

Prevalent examples of such interventions are electrophysiological interventions requiring catheter ablations in the left heart, like atrial fibrillation (AF) ablation. AF ablation patients are currently subjected to anticoagulative treatment with Heparin during the procedure and mostly receive continuous Warfarin therapy before and after the treatment.

Currently, only 10% of all new AF patients are treated by AF ablations, but this fraction is expected to increase vastly with the availability of better ablation methods and devices. Secondly, the number of AF patients is expected to increase considerably due to the aging population and increased survival rates after ischemic myocardial pathologies.

Detecting catheter-induced ischemic strokes with the proposed method and system involves MR imaging of the brain with 2D or 3D DWI sequences and 2D or 3D PWI sequences, and high-resolution 2D or 3D MRA sequences of the head and neck.

An MRI method of DWI produces images wherein stroke-damaged areas of the brain appear as bright, high-intensity portions in a brain scan image. DWI works by producing internal magnetic resonance images of biological tissues weighted with the local characteristics of water diffusion. DWI detects the random movement or “Brownian motion” of water molecules in brain tissue. The rate of movement, between a start point and an end point along a general direction, is a measurable quantity called diffusion. Diffusion in water molecules depends on the kinetic energy and temperature of the molecules. Human tissues have structure such as cell membranes, vascular structures, and axon cylinders, for example, which limit or restrict the amount of diffusion. Healthy active areas of the brain contain much Brownian motion and produce low-intensity images, while in damaged areas of the brain only some of the extracellular water enters the damaged cells due to in-effective ion-pumps, which limits water diffusion and such tissue appears as bright areas of an image. Thus a measurement of diffusion in brain cells may provide an accurate description or indication of the health of brain cells.

Diffusion-weighted images are produced by a pair of strong gradient pulses added to a pulse sequence. The first pulse dephases the spins, and the second pulse rephases the spins if no net movement occurs. If net movement of spins occurs between the gradient pulses, signal attenuation occurs. The degree of attenuation depends on the magnitude of molecular translation and diffusion weighting. The amount of diffusion weighting is determined by the strength of the diffusion gradients, the duration of the gradients, and the time between the gradient pulses.

Without dephasing gradients, the degree of difference in image intensity of an active brain area versus the dim image of an inactive damaged brain area is miniscule. However, as the amount of gradient dephasing increases, more contrast is added to the image. The increased contrast distinguishes the normal, healthy and active brain area as an area of lower brightness and intensity, while the stroke-damaged, not-active brain areas appear as a much brighter area.

DWI is currently the most sensitive way to image an acute infarct, an infarct being the death of tissue due to sudden deprivation of circulating blood. Much of the damage caused by a stroke occurs within a few days after the stroke. However, the damage may also increase over time; days, weeks and even months after the stroke, tissues continue to die.

DWI allows detection of hyper-acute ischemic strokes with very high sensitivity and specificity without use of a contrast agent. A recent review of several studies on DWI in stroke detection found an overall sensitivity of 96.6% and a specificity 100%. Animal models and patient studies have shown that DWI can detect strokes with very high sensitivity within minutes of onset, far exceeding any other imaging method available today. DWI incorporates a contrast mechanism which uses ischemia, the influx of water molecules associated with altered ion flux at the cellular membrane and also cytotoxic edema, the reduction of extracellular water-restricted diffusion. Diffusion can be quantified by calculating apparent diffusion coefficient (ADC) maps at different b-values where b is defined by the formula:

b=γ²G²δ²(Δ−δ/3)

where b is the quantity of dephasing;

γ represents a constant;

G represents gradient amplitude;

δ represents duration of the gradients; and,

Δ represents a leading to trailing edge separation of the gradients.

Lesions detected in diffusion-weighted images describe the non-variable infarcted tissue at the core of the infarction, especially several hours after stroke onset. However, in diffusion-weighted images taken very early after stroke onset, parts of the tissue in the diffusion-weighted lesion can survive.

PWI monitors brain perfusion by measuring vascular transit time, cerebral blood volume, and cerebral blood flow by serial analysis of arterial input. Most methods measure relative blood flow and compare the two hemispheres or the individual lobes of the brain for regional differences.

Vascular transit time and cerebral blood volume can be obtained easily on conventional CT and MR systems with a single bolus injection of contrast material. As the bolus of contrast material passes through the cerebral circulation, a transient increase in attenuation or density occurs. The mean transit time is the time it takes the contrast bolus to pass from the arterial to the venous side of the cerebral circulation.

PWI can indicate cells which are edemic, i.e., no longer metabolically active or alive. In a stroke, there is typically a penumbra surrounding the edemic core in which tissue is not edemic, but perfusion is decreased. This penumbra contains impaired, but possibly salvageable tissue, e.g. by vessel recanalizations. PWI can be performed using dynamic contrast enhancement with gadolinium and gradient-echo imaging. Data from the PWI sequence is used to generate perfusion maps. These perfusion maps typically include cerebral blood flow, cerebral blood volume, mean transit time (MTT) and time-to-peak contrast enhancement maps (Tₘₐₓ).

If the diffusion abnormality matches the area of decreased perfusion, no salvageable ischemic brain tissue is present. If the perfusion abnormality is larger than the area of restricted diffusion, the difference identifies the region of reversible ischemia that can be saved if blood flow is re-established promptly. The DWI and PWI/DWI mismatch picture provides a good and fast approximation as to which tissue may survive and which may not survive.

MRA is a method used to evaluate blood flow. A variety of techniques can be utilized, such as administration of a paramagnetic contrast agent (gadolinium) or using a technique known as “flow-related enhancement”, using 2D or 3D MR time-of-flight sequences in which spins in a plane (of the tissue) are caused to resonate. Images are then generated from resonance signals read out a short time later in adjacent planes. In this manner, only flowing tissue that has flowed from the excited plane to the imaging region, e.g., flowing blood, appears in the image.

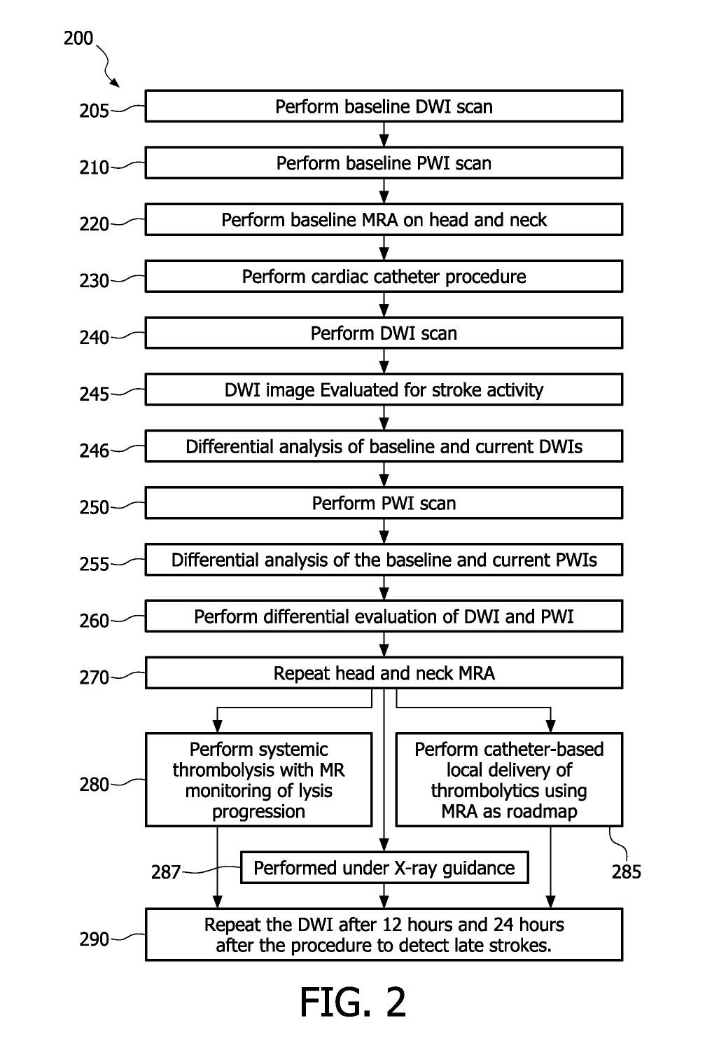

With reference to FIG. 2 and continuing reference to FIG. 1, the present application presents a method 200 and a computer-operable system of diagnosis to diagnose and treat catheterization-induced strokes which incorporates the above-mentioned components of DWI, PWI, and MRA images. The method is directed toward a human patient but the present application does not preclude alternative applications.

The method begins with a step 205 of performing a baseline DWI scan, which comprises producing a diffusion-weighted image and creating a diffusion-weighted image map before the catheterization procedure. This step is optional and the entire method could be performed without performing this step. The baseline DWI image may show prior stroke activity or similar tissue damage.

A baseline PWI scan 210 step generates a perfusion-weighted image map before the catheterization procedure. This step is optional and the entire method could be performed without performing this step. The baseline perfusion-weighted images show preexisting edemic tissue.

A baseline MRA scan 220 step generates MR angiographic images of arteries and veins of the head which may serve as a roadmap in case of a later catheter-based thrombolytic procedure to resolve stroke. The baseline imaging sequences can be performed in any order and can be interleaved.

The next step 230 involves performing a cardiac catheter procedure. The catheter sheath 120 is surgically installed. The sheath is a port through which the catheter is inserted into the patient, typically into an artery. The catheter is then inserted through the sheath and threaded through the vasculature to a target site, e.g., through the arteries to the left ventricle. The catheter may be inserted through the arm at the bend of the elbow in a procedure called the “brachial” approach or the catheter may be inserted at the groin in a procedure called the “femoral” approach or other approaches may be used. The catheter procedure may be performed using appropriate advanced MR imaging and MR catheter tracking techniques. Further MRA images which serve as roadmaps for the catheter procedure may be acquired. To realize catheter tracking, the catheter has a device 130, such as an RF coil, at the tip by which the scanner 20 can localize at least the tip of the catheter in the MRA image. By repeating the imaging procedure or otherwise locating the tip of the catheter periodically, the progress of the catheter can be followed on the MRA images that function analogous to a road map.

When the catheter reaches the target site, the prescribed surgical procedure is performed 230.

Following or during the catheter-based surgical procedure, in a DWI step 240, a DWI sequence is applied by the scanner 20 to generate a diffusion-weighted image. Preferably, the first post-procedure diffusion-weighted image is generated before the sheath is removed. The diffusion-weighted image is evaluated 245 for potential stroke activity. In cases where a baseline diffusion-weighted image was previously taken, a differential analysis of the baseline and current diffusion-weighted images is performed 246, e.g. the baseline and current diffusion-weighted images are subtracted from each other. Particularly during long catheter procedures, stroke detection can be accelerated by pausing the advancement of the catheter from time-to-time, performing another DWI scan, and evaluating the diffusion-weighted image for stroke activity.

If the diffusion-weighted image differential analysis shows a potential stroke, a PWI sequence is performed in a PWI step 250 to generate a current perfusion-weighted image. If a baseline perfusion-weighted image was generated, a perfusion-weighted image differential analysis step 255 is performed to remove signal from any preexisting edemic tissue so that it will not be mistaken as current stroke activity.

The work station 36 includes image processing software which overlays or otherwise combines the MRA image, the DWI image and the PWI image of the brain, to facilitate differential or other comparative analysis 260 of the perfusion-weighted image and diffusion-weighted image. This facilitates an evaluation of the location of the stroke, the extent of the damage and planning of a medical response.

If a stroke is detected, another MRA step 270 is performed. A differential analysis of the current MRA image and the baseline MRA image collected in step 220 is performed to identify vessels that have been stenosed (i.e., narrowed or closed) since the baseline MRA 220. The baseline and current MRA images and the differential MRA image are visualized on the workstation 36 together with the DWI image and PWI image and their respective differential versions to identify and confirm newly clotted or stenosed vessels. Ideally, potential lesions detected in the DWI and PWI images can be associated with corresponding clotted or stenosed vessels. One treatment option is to perform a systemic thrombolysis 280 to break down the blood clot(s) using a pharmacological means to infuse proteins into the bloodstream to dissolve vein or artery blockages. Another treatment option is to dissolve using catheter-based local delivery 285 of the thrombolytics using the MRA images and the DWI and PWI images. Specifically, the catheter is moved through the vasculature using the MRA images as a road map to the site of the clot and a thrombolytic agent is released locally. This enables a large local dose to be delivered to the clot while the total dose to the patient overall remains small. For MR-based catheter guidance 286, additional real-time MR images as well as active tip tracking techniques as described above for the cardiac procedure are used. When the catheter is adjacent and upstream from a target clot or stroke site, the thrombolytics optionally mixed with an MR contrast are dispensed into the blood flow. The distribution of the thrombolytics to the clot can be tracked using a short repeat rate (i.e., fast repetition time) spoiled gradient echo or fast field echo (FFE) scan procedure. Rather than using MR guidance, the catheterization can be performed using X-ray guidance 287.

The DWI step 290 is repeated periodically, such as 12 hours and 24 hours after the catheter-based surgical procedure to detect later strokes. If a stroke is detected, the above-described procedure is repeated. In this manner, the 2D or 3D diffusion-weighted magnetic resonance image of the brain, the 2D or 3D perfusion-weighted magnetic resonance image of the brain, and the high-resolution 2D or 3D magnetic resonance angiographic image of the head and neck are used to detect strokes induced by catheter procedures in the left heart, the aortic arch, the carotid arteries or in cerebral vasculature, or other locations.

This proposed workflow can also be applied to X-ray guided procedures. In that case, the MRA, DWI and PWI images are registered with the X-ray projections that are used to guide the catheter during the catheter-based thrombolysis. However, a very convenient implementation involves procedures performed in combined X-ray and MR (XMR) suites or with MR-guided procedures because the patient here does not have to change modalities and the MRA, DWI, PWI datasets and the catheter positions from the active tip coil 130 are automatically registered. The present workflow has numerous advantages over other methods within the present art such as, but not limited to, presymptomatic stroke detection, asymptomatic stroke detection, and invasive stroke treatment by local delivery of thrombolytics.

The presymptomatic detection of strokes before they become symptomatic offers the possibility of treating the stroke earlier, which is expected to preserve more brain tissue than performing treatment after symptomatic onset. Many patients that need to undergo catheter procedures are sedated and immobilized on the patient bed during and after the procedure. As a consequence, the ability of the patient and clinician to notice neurological disorders is impaired at least for some time after the procedure, which increases the importance of presymptomatic detection. This increases quality of life, reduces the chances of disablement and cost for rehabilitation. Presymptomatic detection is important after catheterization also because sedated and immobilized patients have a reduced chance to notice stroke symptoms early.

Asymptomatic strokes can occur after many types of cardiac catheterizations and can sensitively be detected by DWI. DWI also shows multiple acute lesions, often tiny, cortical and in different vascular territories, distinct from the symptomatic lesion. Detected asymptomatic strokes can be treated, which may otherwise develop into serious strokes. Detected early asymptomatic strokes may also indicate a generally increased post-procedural stroke risk in a patient and could trigger respective preventive treatment.

Stroke treatment, especially invasive treatment by catheter-delivered thrombolytics can be performed with reduced cost, when the patient is still in or in the vicinity of the catheterization unit, and the vascular sheath is still in place and available for use for stroke treatment. Local delivery allows reduced systemic dose of thrombolytics and the associated side effects, such as bleeding. Preferably, the entire procedure is performed in an MR or XMR system. Systemic delivery can also be monitored by DWI, PWI and MRA.

The same applies for the case of a fully MR-guided AF ablation procedure, which is a subject of current research. In both cases, repeated MRI scans can be used to monitor the local delivery and to prove reperfusion of the vasculature and brain tissue.

The application of the workflow proposed herein is of increasing importance due to the expected high number of AF ablation cases and associated risk of acute stroke. However, it is not limited to such interventions but can be applied to all catheter interventions that induce an increased stroke risk. Other applications include coronary angiography, coronary balloon angioplasty, coronary stenting, carotid atherectomy, and angioplasty and stenting in aortic arch coarctation.

The method, system and apparatus described herein may also incorporate a computer-operable means including but not limited to a computer data input means, a computer display terminal for presenting data, a computer memory that may contain a database, and a network connection that may enable the method, system and apparatus to interact on a computer network system including but not limited to the Internet.

Such a method and system are typically performed using computer-operable software instructions and data embedded within a computer hardware memory and run on a computer processor.

The invention has been described with reference to the preferred embodiments. Modifications and alterations may occur to others upon reading and understanding the preceding detailed description. It is intended that the invention be construed as including all such modifications and alterations insofar as they come within the scope of the appended claims or the equivalents thereof. In the claims, any reference signs placed between parentheses shall not be construed as limiting the claim. The word “comprising” does not exclude the presence of elements or steps other than those listed in a claim. The word “a” or “an” preceding an element does not exclude the presence of a plurality of such elements. The disclosed method can be implemented by means of hardware comprising several distinct elements, and by means of a suitably programmed computer. In the system claims enumerating several means, several of these means can be embodied by one and the same item of computer readable software or hardware. The mere fact that certain measures are recited in mutually different dependent claims does not indicate that a combination of these measures cannot be used to advantage.

1. A method of early detection and treatment of catheter-induced ischemic strokes related to performing a catheter procedure, the method comprising:
performing a diffusion-weighted imaging scan on a patient's head to generate a stroke detection diffusion-weighted image;
evaluating the stroke detection diffusion-weighted image for a catheter-induced ischemic stroke; and
optionally repeating the diffusion-weighted imaging scan and the evaluating step periodically, for example, after 12 hours and 24 hours.

2. The method according to claim 1, further including:
performing a perfusion-weighted imaging scan to generate a stroke detection perfusion-weighted image; and
wherein the evaluating step includes evaluating the stroke detection perfusion-weighted image.

3. The method according to claim 2, further including:
performing at least one of a baseline diffusion-weighted imaging scan to generate a baseline diffusion-weighted image and a baseline perfusion-weighted imaging scan to generate a baseline perfusion-weighted image prior to performing the catheter procedure; and
after the catheter procedure, differentially combining the corresponding baseline and the stroke detection images, preferably by performing a differential analysis of the diffusion-weighted images and perfusion-weighted images to evaluate whether a stroke has occurred.

4. The method according to claim 1, further including:
during the catheter procedure, repeating the step of performing the diffusion-weighted imaging scan to generate diffusion-weighted images during the catheter procedure to detect strokes occurring before the catheter procedure is completed.

5. The method according to claim 1, further including:
performing a baseline magnetic resonance angiography scan on the patient's head and neck to generate a baseline magnetic resonance angiographic image;
further performing a magnetic resonance angiography scan to generate an updated magnetic resonance angiographic image before a catheter-based local delivery;
performing a differential analysis of the updated magnetic resonance angiographic image with the baseline magnetic resonance angiographic image to locate a vascular lesion; and
associating this vascular lesion with lesions identified in the diffusion-weighted image and the perfusion-weighted image.

6. The method according to claim 5, further including:
in response to determining that the stroke occurred, performing a catheter-based local delivery of thrombolytics optionally mixed with an MR contrast agent, to treat a stroke site using X-ray or MR based images for catheter guidance and using the updated magnetic resonance angiographic image as roadmap.

7. The method according to claim 2, further including:
combining the stroke detection diffusion-weighted image and the stroke detection perfusion-weighted image to generate a combined image which shows edemic regions and stroke-injured regions, the evaluating step being performed on the combined image.

8. The method according to claim 1, further including:
inserting a catheter configured to dispense thrombolytics into a vascular system;
repeatedly generating real-time images and catheter position measurement to track movement of the catheter through the vascular system to a location adjacent a site of the detected ischemic stroke;
dispensing the thrombolytics, optionally mixed with an MR contrast agent;
performing additional MR imaging scans, such as spoiled gradient echo or fast field echo sequences, to monitor the delivery of the thrombolytic to the ischemic stroke site.

9. A computer-readable medium carrying software for controlling one or more processors to perform the method according to claim 1.

10. A system for the early detection and treatment of catheter-induced ischemic strokes related to performing a catheter procedure, the system comprising:
a magnetic resonance system including a sequence controller for performing a plurality of imaging sequences, and a sequence memory capable of storing at least a magnetic resonance angiography sequence, a diffusion-weighted imaging sequence, and a perfusion-weighted imaging sequence; and
a processor programmed to control the magnetic resonance system to perform the steps of:
performing a catheter tracking procedure to track a catheter during the catheter procedure;
performing the diffusion-weighted imaging sequence to generate a stroke detection diffusion-weighted image; and
from the stroke detection diffusion-weighted image, generating an image for use in analyzing an ischemic stroke.

11. The system according to claim 10, wherein the processor is further programmed to control the magnetic resonance system to perform the steps of:
performing the magnetic resonance angiography sequence on the head and neck to generate a baseline magnetic resonance angiographic image;
further performing the magnetic resonance angiography sequence to generate an updated magnetic resonance angiographic image;
performing differential analysis of the updated magnetic resonance angiographic image and the baseline magnetic resonance angiographic image to highlight a vascular lesion; and
correlating the vascular lesion-highlighted magnetic resonance angiographic image with the diffusion-weighted image.

12. The system according to claim 11, wherein the processor is further programmed to control the magnetic resonance system to perform the step of:
during a catheter-based local delivery of thrombolytics, generating a plurality of real-time images and catheter position measurements by means of a tip coil for catheter guidance, generating MR images to monitor dispensing of thrombolytics, optionally mixed with an MR contrast agent, from the catheter to a stroke site.

13. The system according to claim 10, wherein the processor is further programmed to control the magnetic resonance system to perform the steps of:
performing a baseline diffusion-weighted imaging sequence prior to performing the catheter procedure to generate a baseline diffusion-weighted image;
after or during the catheter procedure, differentially combining the baseline and stroke detection diffusion-weighted images to generate a differential diffusion-weighted image;
displaying the differential diffusion-weighted image to assist in determining if a stroke occurred.

14. The system according to claim 13, wherein the processor is further programmed to control the magnetic resonance system to perform the steps of:
performing the perfusion-weighted imaging sequence to generate a stroke detection perfusion-weighted image; and
combining the stroke detection diffusion-weighted image and the stroke detection perfusion-weighted image to generate a combined image.

15. The system according to claim 14, further including a display:
wherein the processor is further programmed to cause to display a selectable one or more of:
the combined image;
the diffusion-weighted image;
the perfusion-weighted image;
catheter tracking positions and images;
the magnetic resonance angiographic image;
the magnetic resonance angiographic image with catheter positions denoted thereon along with the diffusion-weighted image and the perfusion-weighted image.
